(19) United States
(12) Patent Application Publication
(10) Pub. No.: US 2015/0027218 A1
(43) Pub. Date: Jan. 29, 2015

(54) SECURITY SUBSTRATE, AND METHOD FOR AUTHENTICATION AND FOR REVEALING ATTEMPTS AT FORGERY

(71) Applicants: Sandrine Rancien, Voiron (FR); Celine Desnous, Boulogne Billancourt (FR)

(72) Inventors: Sandrine Rancien, Voiron (FR); Celine Desnous, Boulogne Billancourt (FR)

(73) Assignee: ARJOWIGGINS SECURITY, Boulogne Billancourt (FR)

(21) Appl. No.: 14/235,778

(22) PCT Filed: Jul. 25, 2012
(86) PCT No.: PCT/IB2012/053799

§ 371 Date: May 7, 2014

(30) Foreign Application Priority Data
Jul. 29, 2011 (FR) 11 02391

(51) Int. Cl. G06T 1/00; G01N 33/34; G01N 21/64; G01N 29/04; B42D 25/30; B42D 25/405

(52) U.S. Cl. CPC G06T 1/0042; B42D 25/30; B42D 25/405; G01N 21/64; G01N 29/041; G01N 33/346; G01N 2201/062; G01N 2291/0237; G06T 2201/0201
USPC 73/159; 252/408.1; 252/62.54; 428/343; 283/75; 427/7

(57) ABSTRACT

A security substrate may include at least one area for authentication and/or for revealing attempts at forgery using solvents. The at least one area may include individualized polymer- or copolymer-based particles comprising at least one visible or detectable marker. The polymer or copolymer may be at least partially soluble in solvents used for forgery.

The present application relates to a novel security substrate and to a novel means for authentication and for revealing forgeries or attempts at forgery carried out on sheet documents or self-adhesive complexes, such as labels or visas. The forgeries or attempts at forgery on these documents or complexes are generally carried out by attack with solvents referred to as “solvents used for forgery” with the aim, for example, of forging the printed variable particulars present on these documents or affecting the integrity of the document, for example by detachment of the self-adhesive complex from the support to which it has been applied. The objective of the attempt at detaching the labels or visas is to reuse the latter for fraudulent purposes.

The present application relates more particularly to security substrates in security documents such as self-adhesive complexes for example, which are cellulose-based, for example paper or synthetic, comprising at least one area for authenticating the document or the complex and revealing in a novel manner the attempt at forgery of the document or self-adhesive complex with solvents other than water, in particular nonpolar solvents.

In general, the problem associated with authentication and that associated with revealing forgeries or attempts at forgery are dealt with independently. For example, in the case of documents of cheque paper type, the authentication is mainly carried out by means of patterns printed with a fluorescent ink which are revealed under excitation with UV radiation, whereas the revealing of an attempt at forgery is carried out by means of reagents referred to as “chemical forgery-preventing” reagents, as is described hereinafter.

It is known practice to incorporate into security documents or papers, for example cheque papers, reagents referred to as “chemical forgery-preventing” reagents intended to cause an irreversible modification of the appearance of the paper which is visible to the naked eye, for example through the appearance of colored marks. The objective of these chemical forgeries or attempts at chemical forgery carried out by persons guilty of fraud is generally to partially or totally efface handwriting or personalizing print present on the paper, for example the amount and/or the beneficiary of the cheque.

It is, for example, possible to introduce, during the manufacturing of safety papers for cheques, colorless or weakly colored substances capable of causing, by acid-base or oxidation-reduction chemical reaction or by solubilization, visible colored reactions in the event of forgery of the printed particulars affixed to the paper, with acids, bases, oxidizing agents such as bleach or solvents, which are chemical products that are generally used during attempts at forging writing.

It is, for example, known practice to incorporate, into security papers for cheques, water-insoluble reagents which cause a black-colored reaction by solubilization on contact with “polar” solvents, such as 90-degrees alcohol, and/or water-insoluble reagents which cause a blue-colored reaction on contact with “nonpolar” solvents, such as spirit or mineral spirits known as “white spirit”, which solvents are often used by forgers to attempt to efface the printed variable particulars on documents.

These chemical reagents which are water-insoluble but which dissolve in solvents cannot be incorporated into papers for information-containing pages of a passport or into paper bases for an impregnable visa or label which are to be brought into contact with adhesives. In the context of a passport booklet, the face of security papers for information-containing pages of the passport comprising printed variable particulars is covered with a transparent protective adhesive-coated film which prevents access to said particulars. These adhesive-coated films are generally applied after personalization by cold pressure or by heat-sealing in dedicated lamination devices. Forgers are thus led to attempt to detach this film using pure solvents or commercial products which are formulated based on solvents, for instance mineral spirits known as “white spirit”, a window washer product, etc., for gaining access to and then mechanically or chemically forging the variable particulars present on the security paper under the film. It is unfortunately not possible to incorporate, into these security papers for information-containing pages, the solvent-reactive reagents used in cheque papers and which are capable of revealing a forgery or attempt at detachment of the protective film with nonpolar solvents. This is because these reagents that react to nonpolar solvents, for example based on anthraquinone, have the drawback of reacting over time with the heat-sealable or pressure-sensitive adhesive compositions used to adhesively bond the protective films onto the paper of the information-containing page of a passport. Indeed, the reagents that react to nonpolar solvents react with the solvents, plasticizers, tackifying agents, wetting agents and residual monomers present in the adhesive compositions and can cause premature coloration of the information-containing page of the passport even though there has been no attempt at forgery.

In the context of an application of visa or security label type, the security paper base also receives an adhesive layer, for example by means of a pressure-sensitive permanent adhesive of acrylic nature, and is then applied, after personalization with the information relating to the bearer and to the validity of the visa, onto a paper of the page of a passport reserved for the visa or onto any support or object of another nature to be traced. Forgers are thus led to attempt to detach these visas or labels in order to reuse them fraudently, using pure solvents or commercial products formulated on the basis of solvents, for instance an alkane-based label remover known as “Label Remover”, etc. The solvents used for this purpose are in particular nonpolar solvents, such as toluene, ethyl acetate or trichloroethylene. As in the case of the page containing variable information of a passport, it is unfortunately not possible to incorporate, into the security papers for paper or synthetic labels, solvent-reactive reagents capable of revealing a forgery or an attempt at detachment of these labels with nonpolar solvents, since these reagents that react to nonpolar solvents, based in particular on anthraquinone, have the drawback of reacting over time with the pressure-sensitive adhesive compositions used to attach these labels.

Document FR 2 675 742 describes a security document comprising a substrate and its protective film which is covered with a transparent adhesive layer. Printing is carried out between the substrate and the adhesive with an ink containing a hydrocarbon-soluble dye. In the event of an attempt at attacking the adhesive layer in order to detach the film by means of a hydrocarbon, the printing dye dissolves and marks the film and the substrate, thus revealing the attempt at forgery. However, such printed inks also have the major drawback of reacting over time with the adhesive layer of the protected film such that they incorrectly reveal an attempt at forgery.

In the context of the visa or label application, in order to solve this problem of interaction between the adhesive compositions and the reagents that are reactive to nonpolar solvents which are in particular used for cheques, it has been proposed by the applicant to place a barrier layer between the adhesive and the paper base of the substrate of the label comprising the solvent-reactive reagents in order to prevent any migration of solvents, plasticizers, tackifying agents, wetting agents and residual monomers present in the adhesive compositions toward the paper comprising the solvent-reactive reagent, so as to prevent any premature solubilization of the reagents and therefore the appearance of marks which may be incorrectly identified as corresponding to an attempt at forgery. Although this solution is effective, it has the drawback of adding an additional coating step to the process for manufacturing the visa or the label and also of modifying the adhesive properties of the adhesive label owing to less penetration of the adhesive into the substrate during coating thereof. As a result of this, the adhesive strength of the label on the security paper is reduced and the detachment of the label therefrom is facilitated accordingly.

The invention aims to solve the drawbacks of the prior art by providing a novel means for authenticating a security document and revealing forgeries or attempts at forgery.

The invention therefore relates to cellulose- and/or polymer-based printable security substrates for single-layer sheet documents, for instance an inside page of a passport, or for multilayer sheet documents, for instance identification cards, or for self-adhesive complexes of the security visa or label type, these substrates comprising individualized particles enabling authentication of said substrate while at the same time being capable of revealing a forgery or an attempt at forgery with solvents, without using the solvent-reactive reagents such as the water-insoluble but solvent-soluble dyes or pigments of the prior art.

The cellulose- and/or polymer-based security substrates according to the invention can be prepared as a sheet on a paper machine from a fibrous suspension, or by (co)extrusion and/or bi-stretching of a structure made of synthetic material, in particular based on a polyolefin, for example polyethylene.

The object of the present invention is in particular to make security documents in contact with adhesives, such as the information-containing pages of a passport and visas, authenticable while at the same time making them more difficult to forge in terms of the variable particulars or to detach from the support to which they have been applied for the purpose of fraudulent reuse of the latter, by means of individualized particles, which make it possible both to provide increased revealing of attempts at forgery with solvents, whether this forgery is carried out by touch or by soaking, and also to authenticate the security document.

The object of the present invention is achieved by providing a security substrate comprising at least one area for authentication and/or for revealing attempts at forgery using solvents, in particular nonpolar solvents, characterized in that said area comprises individualized polymer- or copolymer-based particles comprising at least one marker, and in that said polymer or copolymer is at least partially soluble in solvents used for forgery. Preferably the individualized polymer- or copolymer-based particles of the security substrate comprising at least one marker according to the invention do not comprise substances capable of interacting with adhesive compositions. For example, the individualized polymer- or copolymer-based particles do not include compounds based in particular on anthraquinone which are capable of reacting over time with the plasticizers, tackifying agents, wetting agents and/or residual monomers present in adhesive compositions, in particular the pressure-sensitive or heat-sealable adhesives which may be in contact with the substrate.

Advantageously, the area for authentication and/or for revealing attempts at forgery may correspond only to a part of the substrate, for example that on which the variable particulars are inscribed, so as to limit costs. Alternatively, the area for authentication and/or for revealing attempts at forgery may correspond to the entire surface of the substrate such that any attempt at forgery is detected. The area for authentication and/or for revealing attempts at forgery forms an integral part of the security substrate. The term “integral part of the security substrate” is intended to mean a part which shares with the rest of the substrate structuring components, i.e. fibers for the cellulose-based substrates and synthetic materials, for example (co)extruded and/or bi-stretched synthetic materials, for the polymer-based substrates.

In particular, the area for authentication and/or for revealing attempts at forgery does not comprise or does not correspond to a layer removable by scratching which masks printed information.

According to one particular embodiment of the invention, the area for revealing attempts at forgery using solvents is placed in the body and/or at the surface of the security substrate.

When the security substrate is fibrous, the individualized particles can be mixed with paper-making fibers. It is possible for the individualized particles not to be coated in a latex.

Advantageously, the area for authentication and/or for revealing attempts at forgery does not mask printed information, unlike, for example, a layer removable by scratching.

In particular, the individualized particles can be directly dispersed in the body of the substrate or of one of the plies of the substrate and/or applied at the surface of the substrate by means of a layering, coating, impregnating or printing process which makes it possible both to authenticate the substrate and to reveal an attempt at forgery by means of a solvent attack by the surface of the substrate or via the section of the security document whatever the area of attack. These individualized polymer- or copolymer-based particles comprising at least one marker can also be introduced into the substrate via intermediate carriers such as security elements, for instance paper or polymer strips, security yarns, or small fragments of paper or of plastic termed planchettes, into which said individualized particles will have been incorporated beforehand.

According to one particular embodiment of the invention, the individualized particles comprise a single type of polymer or a mixture of polymers of different solubilities in solvents used for forgery. In particular, the individualized particles comprise polymers of polystyrene, of polycarbonate, of polyvinyl acetate, and/or other polymers soluble in solvents, in particular in toluene, ethyl acetate or trichloroethylene, which are very widely used for the forgery of the particulars printed on the substrate or for detaching labels.

The individualized particles according to the invention consist of a polymer-based matrix. The term “polymer” is intended to mean a homopolymer, a copolymer or a mixture of polymers of different natures which have chemical solubility in the solvents normally used for the forgery or the fraudulent reuse of documents. The individualized particles may, for example, consist of a mixture of several polymers known to be soluble in different solvents used for forgery. The polymers retained will preferably be polystyrene, polycarbonate and polyvinyl acetate which dissolve in nonpolar solvents such as toluene very widely used for detaching protective films and labels.

Among these solvents used for forgery, mention may be made of polar solvents of alcohol type, such as methanol, ethanol, etc., ketones, such as acetone, etc., esters, such as ethyl acetate, etc., halogenated solvents, such as dichloromethane, and amines, and nonpolar solvents, in particular hydrocarbon-based nonpolar solvents, of aliphatic type, such as hexane, heptane, etc., aromatic solvents such as toluene, mineral spirits known as “white spirit”, etc.

The individualized polymer-based particles used in the substrate according to the invention can have various forms or sizes. They will in particular be introduced into the body and/or at the surface of the security substrate in the form of grains, solid or hollow spheres, fibers, disks, lamellae or needles.

According to one particular embodiment of the invention, the individualized polymer-based particles can be functionalized with carboxyl, amine, hydroxyl, sulfate, etc., functions in order to promote their attachment in the substrate.

The individualized particles have a uniform or dispersed size distribution. The individualized particles in the form of lamellae or of needles preferably have a larger dimension less than 200 μm and the individualized particles in the form of a sphere preferably have an average diameter between 1 and 200 μm, so as to rapidly dissolve in the solvents used by forgers. Experiments have shown that satisfactory results are obtained for such individualized-particle sizes. Even faster solubilizations can, however, be obtained when the largest dimension of individualized particles is less than 50 μm.

It has been observed that a security substrate comprising a number of individualized particles per cm² of between 5 and 1000 enables both satisfactory authentication of the substrate and good revealing of attempts at forgery.

The invention extends to a process for authentication of the security substrate or of a security document including said substrate, comprising the steps consisting in:
providing individualized polymer- or copolymer-based particles comprising at least one marker, said polymer or copolymer being at least partially soluble in solvents used for forgery, said marker being visible or detectable, a visible marker being in particular iridescent, luminescent, fluorescent, phosphorescent, photochromic, thermochromic, a visible marker being visible in particular under excitation by UV, visible or infrared radiation or by means of specific optics, a detectable marker having in particular electrical, magnetic or electromagnetic properties, and in particular being detectable under excitation by visible or infrared electromagnetic radiation, radiofrequency vibration or at an ultrasonic frequency, and
verifying that said marker is visible or detectable in such a way as to authenticate the security substrate or the security document.

It is therefore understood that, if the marker is visible or detectable, this means that the security substrate or the security document is authentic.

In the event of an absence of visible or detectable markers, and therefore of an absence of marked individualized particles in or on the substrate or the document, it can be concluded therefrom that the security substrate or the security document is a forgery, i.e. that it is not authentic or that it has been forged in solvents.

The markers according to the invention can be authenticable in various ways:
visible to the naked eye under daylight and without particular accessory, or
“semi-visible”, i.e. visible to the naked eye but using an accessory for example having a specific illuminant and/or specific optics, or
automatically detectable with a detector capable of detecting a specific property of the marker, for example a magnetic, electromagnetic, electrical, etc., property, for example detectable under excitation by visible or infrared electromagnetic radiation, radiofrequency vibration or at an ultrasonic frequency.

Among the visible markers, markers visible to the naked eye under daylight and without accessory are therefore distinguished from markers visible with an accessory, termed “semi-visible”.

Among the markers visible to the naked eye under daylight and without accessory, mention may be made of iridescent pigments, colored particles, liquid crystal polymers, liquid crystals, metal deposits and variable optical-effect elements.

Among the markers visible with an accessory such as a lamp or LED (light emitting diode) having a suitable UV (ultraviolet), visible or IR (infrared) excitation wavelength, mention may be made of luminescent, fluorescent, phosphorescent or photochromic dyes or pigments. Among the markers visible with a heating accessory, mention may be made of thermochromic pigments.

Among the markers which can be revealed with a detector, mention may be made of markers which have magnetic properties, such as certain alloys, ferromagnetic properties with soft or hard magnetism properties, electromagnetic properties, markers which have luminescent properties in the UV, visible or IR range, markers which have electrical, in particular conducting, properties, and markers detectable by radiofrequency vibration or ultrasound.

According to one particular embodiment of the invention, the marker is contained inside and/or placed at the surface of the individualized polymer particles and/or mixed with the polymer or copolymer constituting said particle. For example, a particle in the form of a hollow sphere can contain a marker at the surface of the sphere, in its shell, and/or inside the sphere, etc.

The markers can be included in the particle by means of a grafting, coating, encapsulating or aggregating process. Alternatively, the markers can be placed at the surface of the particle by means of a process of adsorption or of dyeing on the individualized particles. Finally, the markers can be mixed into the body with the polymer material before the particle individualization step.

In one particular embodiment of the invention, the security substrate comprises both first individualized polymer- or copolymer-based particles comprising at least one first marker for authentication of said substrate, said polymer or copolymer being at least partially soluble in solvents used for forgery, and second individualized polymer- or copolymer-based particles comprising at least one second marker, which may be different than the first, also for authentication of said substrate, said polymer or copolymer of said second particles being insoluble in solvents used for forgery. By mixing first individualized particles which may no longer be authenticable after forgery with solvents and second individualized particles which will remain authenticable via the second marker after forgery with solvents, it is advantageously possible to distinguish between a nonauthentic substrate (therefore which will reveal neither first individualized particles comprising the first marker, nor the second individualized particles comprising the second marker) and a substrate forged with solvent (therefore which will no longer reveal the first individualized particles comprising the first marker, but will still reveal the second individualized particles comprising the second marker).

The invention extends to a security document comprising the security substrate and including in particular other security elements which do not take the form of individualized polymer-based particles comprising a marker.

The document can in particular comprise other authentication and forgery-preventing security elements used alone or in combination, for instance:
liquid crystal pigments, in particular in printed or coated form,
an interferential multilayer film,
a planar structure with variable optical effects based on interferential pigments or liquid crystals,
a birefringent or polarizing layer,
a planar diffraction structure,
a partially reflective, refractive planar element,
a transparent lenticular planar grating,
a layer with variable optical effect based on interferential pigments or liquid crystals,
a flat security element of relatively small format, such as a planchette,
security fibers with a circular or rectangular cross section, which are in particular luminescent, metallic, magnetic,
a flat security thread, which is in particular metalized or holographic with areas potentially demetalized forming a pattern or text,
a hot-laminated or cold-laminated planar track, which is in particular metalized, and holographic with demetalized areas forming a pattern or text.

The invention also extends to a security document including the security substrate and comprising an adhesive layer such as a visa or a security label.

The invention further extends to a process for revealing forgery of a security substrate or of a document comprising said security substrate, comprising the steps consisting in:
providing first individualized polymer- or copolymer-based particles comprising at least one first marker, said polymer or copolymer being at least partially soluble in solvents used for forgery, said first marker being visible or detectable, a visible marker being in particular colored, iridescent, luminescent, fluorescent, phosphorescent, photochromic, thermochromic, a visible marker being visible in particular under excitation by UV, visible or infrared radiation or by means of specific optics, a detectable marker having in particular electrical, magnetic or electromagnetic properties, and in particular being detectable under excitation by UV, visible or IR electromagnetic radiation, radiofrequency vibration or at an ultrasonic frequency, and
verifying that said first marker is invisible or undetectable so as to reveal an attempt at forgery of said substrate or of said document using solvents, or so as to conclude that the security substrate or the security document is not authentic, or
verifying that said first marker is visible but has a different appearance or is detectable but exhibits a different signal so as to reveal an attempt at forgery of said substrate or of said document using solvents.

It is understood that, if the marker is invisible or undetectable, it may be concluded therefrom that there has been an attempt at forgery or that the security substrate or the security document is not authentic.

It is understood that, if the first marker is visible but has a different appearance, it may be concluded therefrom that there has been an attempt at forgery on an authentic security substrate or security document.

In the case where the marker is invisible and undetectable following an attempt at forgery using solvents, the revealing of the forgery is not linked to the appearance of colored marks on the substrate owing to solubilization of the solvent-soluble reagents, but is linked to the disappearance of said individualized particles which will no longer be visible to the naked eye with or without the aid of an accessory or no longer be detectable with the appropriate detection system, thus indicating that there has been forgery or an attempt at forgery in one or more solvents. The revealing of the attempt at forgery is based on the fact that, subsequent to the contacting of said security substrate with solvents used by forgers, the individualized polymer-based particles present in and/or at the surface of this substrate are partially or totally dissolved and that the markers that they contain in the body or at the surface will have diffused in the substrate or will have migrated in the solvent used for forgery. During the verification of the substrate or of the document, the absence of marked individualized particles or the modification of the form, color or property thereof, for example fluorescence, will make it possible to characterize the fraud. In the case of the absence of marked individualized particles, if no other element enables authentication of the document, the absence of marked individualized particles can also be linked to the fact that the document is not authentic and no longer to forgery of the document.

According to one particular embodiment, the authentication process according to the invention also comprises the steps consisting in:
providing second individualized polymer- or copolymer-based particles comprising at least one second marker, said polymer or copolymer of said second individualized particles being insoluble in solvents used for forgery and said second marker being visible or detectable,
verifying that said second marker is visible or detectable so as to reveal an attempt at forgery of said substrate or of said document using solvents.

In this case, the substrate or the document is necessarily authentic since the second marker is visible or detectable.

If the first marker is invisible or undetectable and the second marker is visible or detectable on or in the security substrate or the security document comprising said substrate, it can be concluded therefrom that the substrate or the document has been forged in solvents but that it is authentic.

On the other hand, if the second marker is invisible or undetectable, whatever the state of the first marker, on or in the security substrate or the security document comprising said substrate, it can be concluded therefrom that the substrate or the document is not authentic but that it is a forgery.

Finally, if the first and second markers are visible or detectable on or in the security substrate or the security document comprising said substrate, it can be concluded therefrom that the substrate or the document is authentic and that it has not been forged.

The invention also extends to a process for manufacturing a security substrate or a security document, characterized in that it comprises:
a step consisting in introducing the individualized polymer- or copolymer-based particles comprising at least one marker into the body of the substrate or of the document, and/or
a step consisting in applying said individualized polymer- or copolymer-based particles comprising at least one marker at the surface of said substrate or of said document, in particular by means of a surfacing, layering, coating, impregnating or printing step.

According to one particular embodiment, the manufacturing process according to the invention comprises a step consisting in randomly dispersing said individualized polymer- or copolymer-based particles comprising at least one marker in a fibrous suspension before or during formation of said substrate in sheets, on a paper machine, so as to randomly introduce said individualized particles into the body of the substrate or the document.

According to one particular embodiment, the manufacturing process according to the invention comprises a step consisting in randomly dispersing said individualized polymer- or copolymer-based particles comprising at least one marker in an aqueous solution optionally supplemented with a wetting agent and with a binder and in applying this mixture in a strip for example via a spray just after the formation of the sheet but before the pressing of said substrate on a paper machine, so as to introduce said randomly distributed individualized particles at the surface of the substrate or of the document, for example in the form of a strip.

According to a further particular embodiment, the manufacturing process according to the invention comprises a step consisting in randomly mixing said individualized polymer- or copolymer-based particles comprising at least one marker with a structure made of synthetic material, in particular based on polyethylene, having voids or cavitations, to which mineral fillers have optionally been added, before steps of coextrusion and/or of bi-stretching of said structure made of synthetic material and the creation of voids in the structure made of synthetic material with cavitations, or in randomly applying, at the surface of the substrate, said individualized polymer- or copolymer-based particles comprising at least one marker to said structure made of synthetic material, in particular based on polyethylene, having voids, after steps of coextrusion and/or of bi-stretching of said structure made of synthetic material.

According to a further particular embodiment, the manufacturing process according to the invention comprises the following particular steps: placing an adhesive layer on the security substrate and providing said structure made of synthetic material based on polyethylene having voids with a density of less than 0.6 g/cm³, and providing said structure made of synthetic material such that it has a cohesive strength of less than 4 Newtons.

Advantageously, such a manufacturing process makes it possible to obtain an adhesive-coated security substrate or document of which the density and the cohesive strength of the front piece made of synthetic material are such that any attempt at dry detachment, i.e. without solvent, will lead to tearing and/or at least partial delamination of the substrate or of the document, thus revealing the attempt at forgery by mechanical detachment.

### EXAMPLES

The exemplary embodiments described in greater detail hereinafter are given only by way of indicating example which in no way limits the invention.

According to a first exemplary embodiment of the invention, individualized polymer-based particles and more particularly polystyrene spheres 4 microns in diameter and which are visible to the naked eye are synthesized using a dedicated accessory. A specific luminescent marker excitable at certain wavelengths in the visible or UV range and fluorescent in the visible range is attached to the polystyrene constituting the spheres so as to enable detection of these spheres which are normally invisible to the naked eye, using a detection system with appropriate illumination, filters and magnifying devices. These marked polystyrene spheres are introduced into and dispersed in the paper pulp before the sheet formation on the paper machine so as to obtain about 10 spheres per cm². The security paper or substrate obtained is then coated with a permanent pressure-sensitive adhesive of acrylic type which is covered with a protective silicone film.

Forgery simulations and more particularly tests consisting of detachment of these self-adhesive labels after 24 hours of application on a support are carried out by chemical attack by immersion for a few seconds of the adhesive-coated paper applied to its support using solvents, in particular of toluene or trichloroethylene type, which are very effective for detaching self-adhesive labels without destroying the front piece of the label.

After 30 seconds of soaking in toluene, the adhesive labels detach without any damage to their detection support and can be reattached to another support without showing any apparent evident forgery to the naked eye. On the other hand, observation of the front face of the label with the appropriate detector shows that the spheres marked with luminescent markers and initially introduced into the paper of the front face of the label are no longer detectable; forgery or the fact that the substrate is not authentic is therefore clearly revealed.

This example makes it possible to demonstrate the fraudulent reuse of an authentic adhesive-coated paper of visa type that has been detached and reattached to another passport or the use of nonauthentic adhesive-coated paper.

In the case of forgery, the individualized polymer-based particles comprising the luminescent marker, initially present in the paper, are no longer visible or no longer detected when the document is checked, thus revealing the chemical fraud in solvents. This is because the polystyrene spheres which are soluble in solvents such as toluene have been completely dissolved.

In addition, with this security substrate or adhesive-coated paper according to the invention, the constituents of the acrylic adhesive do not react over time with the individualized polymer-based particles so as to reveal marks and lead to incorrect detections of an attempt at forgery, as is the case with the prior art.

According to a second exemplary embodiment of the invention, individualized particles which are fluorescent under UV radiation at a wavelength of 365 nm, of undefined form, with their largest dimension being a few hundred micrometers, are manufactured in a fluidized bed from powdered fluorescent pigments of 3 to 5 micrometers aggregated with a binder of low-molecular-weight latex type which is partially soluble in the solvents. These fluorescent individualized particles are introduced discretely and dispersed in the paper pulp, as a mixture with flat elements of security planchette type which are visible to the naked eye but with an accessory, i.e. fluorescent under UV radiation at 365 nm, before the formation of the sheet on the paper machine, so as to obtain a substrate or paper having an individualized-particle density of about one hundred per cm² and a planchette density of about a few hundred per m².

Under the radiation of a UV lamp at a wavelength of 365 nm, these fluorescent individualized particles appear distinctly on the security sheet and are dispersed according to a random pattern similar to a “starry sky”. The security planchettes are also visible under this irradiation wavelength.

Forgery simulations and more particularly tests for forgery of handwriting produced with a permanent black marker on this paper are carried out via chemical attack by touch with a cotton bud soaked in solvent, in particular of ketone type, which is very effective for effacing this type of writing.

After a few gentle moisture rubbing operations, the paper no longer shows any trace of the written particulars and new particulars can be re-written on the paper without showing any apparent evident forgery to the naked eye. On the other hand, observation of the paper under a UV lamp at a wavelength of 365 nm shows that the fluorescent individualized particles no longer have their initial shape and have diffused, whereas the planchettes are still detected, characterizing evident forgery on an authentic security paper. In this example, the fluorescent individualized particles were aggregated by means of a polymer having properties, after drying, of solubilization in solvents used for forgery, such that, in the event of contact with these solvents, the aggregates of fluorescent pigments are dissolved and the pigments are scattered at the surface of the security sheet.

If UV radiation is applied to the sheet thus modified, it is no longer possible to distinguish the distinct fluorescent spots, but diffuse fluorescent areas of lower intensity, instead. In this case, the attempt at forgery is revealed by checking the modification of the visual appearance of the marker after the fraud.

This evident forgery can be detectable automatically by sorting machines comprising a module for illumination under UV at 365 nm coupled to a CCD camera allowing acquisition of the image which, once processed and analyzed, makes it possible to say whether the two UV elements are detectable and exhibit a signal identical to the single expected for an unforged authentic document.

According to a third exemplary embodiment of the invention, individualized particles made of polystyrene and of polyethylene in the form of spheres 15 microns in diameter, which are visible to the naked eye but with an accessory, are respectively synthesized. A specific luminescent marker excitable at certain wavelengths in the UV range or the visible range and fluorescent in the visible range is attached to the polystyrene spheres and another specific luminescent marker, still excitable in a UV or visible wavelength range and emitting at other wavelengths in the visible range is attached to the polyethylene spheres. These spheres as a mixture are normally invisible to the naked eye, but visible using a detection accessory with the appropriate illumination, filters and magnifying devices. These marked polystyrene and polyethylene spheres are introduced into and dispersed in a layering slip which is subsequently coated, by air life, onto a face of a bi-stretched synthetic polyethylene support, so as to obtain approximately fifteen individualized particles per cm². The synthetic security substrate obtained is subsequently coated on its other face with a permanent pressure-sensitive adhesive of acrylic type and covered with a silicone protector.

Forgery simulations and more particularly tests for detachment of these self-adhesive labels after 24 hours of application on a support are carried out by chemical attack by immersion for a few seconds of the adhesive-coated label applied to its support using solvents, in particular of toluene or trichloroethylene type, which are very effective for detaching self-adhesive labels without destroying the front piece of the label.

After 30 seconds of soaking in toluene, the adhesive labels detach without any damage to their support and can be reattached to another support without showing any apparent evident forgery to the naked eye. On the other hand, observation of the front piece of the label with the appropriate accessory shows that the marked polystyrene spheres are no longer visible, whereas the marked polyethylene spheres are visible; chemical forgery on an authentic security substrate is therefore revealed. This is because the first polystyrene spheres are soluble in the solvents used for forgery, whereas the second polyethylene spheres are not.

According to a fourth exemplary embodiment of the invention, individualized polystyrene particles in the form of hollow spheres, the average diameter of which after sorting is about 100 μm, are synthesized. A marker excitable by UV radiation and emitting a fluorescent light in the blue range is encapsulated in these spheres. These marked polystyrene spheres are introduced into and dispersed in the paper pulp before it is formed into sheets on the paper machine, so as to obtain a paper which has an individualized-particle density of about one hundred per cm². The security paper obtained is subsequently coated with a permanent pressure-sensitive adhesive of acrylic type.

Under excitation by UV radiation, these particles appear fluorescent in a discrete and well-individualized manner with a clear shape on the security sheet in which they are randomly dispersed.

Forgery simulations and more particularly the tests for detachment of these self-adhesive labels after 24 hours of application on a support are carried out by chemical attack by immersion for a few seconds of the adhesive-coated label applied to its support, using solvents in particular of toluene or trichloroethylene type, which are very effective for detaching self-adhesive labels without destroying the front piece of the label.

After 30 seconds of soaking in toluene, the adhesive labels detach without any damage to their support and can be reattached to another support. Nevertheless, after the attempt at forgery, under excitation with UV radiation, the fluorescence of the particles will appear diffusely in the security substrate.

According to a fifth exemplary embodiment of the invention, individualized spherical particles a few tens of micrometers in diameter, consisting of solid beads composed of a first solvent-insoluble polymer and coated with a layer of second solvent-soluble polymer, and more particularly polyethylene beads with a polystyrene coating, are manufactured. A first fluorescent marker is mixed with the particle core polymer and emits a red fluorescence under UV excitation at 365 nm and a second fluorescent marker is introduced into the polystyrene coating, constituting the exterior of the particle, and emits a blue fluorescence under UV excitation at 365 nm. Moreover, the interface between the polystyrene coating of the particle and the solid polyethylene bead comprises a substance which screens out UV radiation at 365 nm, such that the fluorescence of the first marker is not visible when the external coating of the particle is not damaged, only the fluorescence of the second marker being observable under UV excitation at 365 nm.

These fluorescent individualized spherical particles are introduced in a discrete and dispersed manner into the paper pulp, before it is formed into sheets on the paper machine, so as to obtain a substrate or paper having an individualized-particle density of about one hundred per cm².

Under the radiation of a UV lamp at a wavelength of 365 nm and by means of a magnifying device, these individualized particles appear distinctly and randomly on the security sheet in the form of spots which have a blue fluorescence.

Forgery simulations are more particularly tests for forgery of handwriting produced with a permanent black marker on this paper are carried out by chemical attack by touch with a cotton bud soaked with solvent, in particular of ketone type, which is very effective for effacing this type of writing.

After a few gentle moist rubbing operations, the paper no longer shows any trace of the written particulars and new particulars can be rewritten on the paper without showing any apparent evident forgery to the naked eye. On the other hand, observation of the paper under a UV lamp at a wavelength of 365 nm and with a magnifying device shows a different fluorescence of the individualized particles, characterizing evident forgery on an authentic security paper.

In this example, the polystyrene coating of the spherical particles dissolved on contact with the solvent, entraining the second fluorescent marker, but the polyethylene bead remains intact as does the first marker. If UV radiation is applied to the sheet thus modified, it is no longer possible to observe the blue fluorescence of the second marker of the individualized particles, but the red fluorescence of the first marker is instead observed.

In this case, the attempt at forgery is revealed through the observation of another fluorescence color after the fraud.

According to a sixth exemplary embodiment of the invention, security planchettes visible to the naked eye under UV excitation at 365 nm, comprising a written particular formed by polymer-based individualized particles which are red fluorescent under UV excitation at 365 nm, on a background which is yellow fluorescent under this same excitation, are manufactured. The fluorescent background like the written particular formed by the fluorescent particles are applied to the support of the planchettes by heliographic printing. The individualized particles are more particularly polystyrene beads with a fluorescent marker, which are visible to the naked eye under UV radiation with a magnifying accessory.

These fluorescent planchettes are introduced discretely into and dispersed in the paper pulp before it is formed into sheets on the paper machine, so as to obtain a substrate or paper having a planchette density of about a few hundred per m². The security paper or substrate obtained is subsequently coated on one of its faces with a permanent pressure-sensitive adhesive of acrylic type, and then covered with a protective silicone film.

Under the radiation of a UV lamp at a wavelength of 365 nm, the red and yellow fluorescences of the security planchettes present in the substrate are visible to the naked eye.

Forgery simulations and more particularly tests for detachment of these self-adhesive labels after 24 hours of application on a support are carried out by chemical attack by immersion for a few seconds of the adhesive-coated paper applied to its final support, using solvents, in particular of toluene or trichloroethylene type, which are very effective for detaching self-adhesive labels without destroying the front piece of the label.

After 30 seconds of soaking in toluene, the adhesive labels detach without any damage to their detection support and can be reattached to another support without showing any apparent evident forgery to the naked eye. On the other hand, observation of the front face of the label under UV excitation at 365 nm shows that the fluorescent planchettes initially introduced into the paper no longer exhibit the red fluorescence of the individualized particles constituting the written particular, only the yellow fluorescent background being observable, proving that there has been forgery of the document. This example makes it possible to demonstrate the fraudulent reuse of an authentic adhesive-coated paper of visa type which has been detached and reattached to another passport.

In the case of forgery, the polystyrene beads have been dissolved in the solvent used for the detachment of the label, entraining the red fluorescent marker, which is therefore no longer visible under UV excitation at 365 nm.

According to a seventh exemplary embodiment of the invention, paramagnetic microspheres having a diameter of 0.9 to 1.8 microns are manufactured with iron oxide encapsulated in the polystyrene spheres functionalized with carboxylic functions so as to prevent agglomeration problems. These magnetic individualized particles are introduced into and dispersed in a layering slip which is subsequently coated, by air life, onto a face of a bi-stretched synthetic polyethylene support, so as to obtain approximately fifteen individualized particles per cm². The synthetic security substrate obtained is subsequently coated onto its other face with a permanent pressure-sensitive adhesive of acrylic type and covered with a silicone protector.

Using a dedicated detection system, these magnetic individualized particles randomly dispersed at the surface of the substrate emit a magnetic signal that it is possible to detect.

Forgery simulations and more particularly tests for detachment of these self-adhesive labels after 24 hours of application on a support are carried out by chemical attack by immersion for a few seconds of the adhesive-coated label applied to its support, using solvents, in particular of toluene or trichloroethylene type, which are very effective for detaching self-adhesive labels without destroying the front piece of the label.

After 30 seconds of soaking in toluene, the adhesive labels detach without any damage to their support and can be reattached to another support without showing any apparent evident forgery to the naked eye. On the other hand, detection of the magnetic signal by means of the dedicated detection system shows a different response of the magnetic pigments compared with the magnetic signal of the polymer-based magnetic individualized particles before forgery. This variation in the signal thus proves that there has been chemical forgery on an authentic substrate.

1-21. (canceled)

22. A security substrate comprising:
at least one area for authentication and/or for revealing attempts at forgery using solvents, wherein the at least one area comprises individualized polymer- or copolymer-based particles comprising at least one visible or detectable marker, and wherein the polymer or copolymer is at least partially soluble in solvents used for forgery.

23. The security substrate of claim 22, wherein the solvents are nonpolar.

24. The security substrate of claim 22, wherein, if the marker is not visible or detectable, the security substrate is not authentic and/or has been forged.

25. The security substrate of claim 22, wherein the at least one area for authentication and/or for revealing attempts at forgery using solvents is positioned in a body and/or at a surface of the security substrate.

26. The security substrate of claim 22, wherein the individualized particles comprise a single type of polymer or a mixture of polymers having differing solubilities in solvents used for forgery.

27. The security substrate of claim 22, wherein the individualized particles comprise at least one polymer which is soluble in polar or nonpolar solvents.

28. The security substrate of claim 27, wherein the at least one polymer is soluble in nonpolar solvents.

29. The security substrate of claim 28, wherein the at least one polymer is soluble in hydrocarbon-based nonpolar solvents.

30. The security substrate of claim 29, wherein the at least one polymer is soluble in at least one of alcohols, ketones, esters, halogenated solvents, amines, aliphatic solvents, aromatic solvents and mineral spirits.

31. The security substrate of claim 22, wherein the individualized particles comprise polystyrene, polycarbonate, polyvinyl acetate, and/or other polymers which are soluble in at least one of toluene, ethyl acetate and trichloroethylene.

32. The security substrate of claim 22, wherein the at least one marker is contained inside and/or is placed at a surface of the individualized polymer particles and/or is mixed with the polymer or copolymer of the particles.

33. The security substrate of claim 22, wherein the at least one visible or detectable marker is iridescent, luminescent, fluorescent, phosphorescent, photochromic, and/or thermochromic, and wherein the at least one visible or detectable marker is visible under excitation by radiation in the ultraviolet spectrum, infrared spectrum, or visible spectrum, or by specific optics; or wherein the at least one visible or detectable marker has electrical, magnetic, and/or electromagnetic properties and is detectable under excitation.

34. The security substrate of claim 33, wherein the at least one visible or detectable marker is detectable by radiofrequency vibration or ultrasound.

35. The security substrate of claim 22, further comprising second individualized polymer- or copolymer-based particles comprising at least one second marker, wherein the polymer or copolymer of the second individualized particles is insoluble in solvents used for forgery and the at least one second marker is visible or detectable.

36. The security substrate of claim 22, wherein the security substrate is cellulose- and/or polymer-based, and is prepared as a sheet on a paper machine from a fibrous suspension, or by extrusion and/or bi-stretching from a structure made of synthetic material.

37. The security substrate of claim 36, wherein the synthetic material is a polyolefin.

38. The security substrate of claim 37, wherein the polyolefin is polyethylene.

39. The security substrate of claim 22, wherein the at least one area for authentication and/or for revealing attempts at forgery using solvents is not a printed-information-masking layer removable by scratching.

40. A security document comprising a security substrate as claimed in claim 22 and further comprising other security elements.

41. The security document of claim 40, wherein the security document comprises an adhesive layer.

42. The security document of claim 41, wherein the adhesive layer comprises a visa or a security label.

43. A method for authenticating a security substrate as claimed in claim 22, comprising:
verifying whether the at least one marker is visible or detectable to authenticate the security substrate or a security document containing the security substrate,
wherein the individualized polymer- or copolymer-based particles comprise at least one marker, the polymer or copolymer being at least partially soluble in solvents used for forgery,
wherein the at least one marker is visible or detectable, and
wherein the at least one marker is irridescent, luminescent, fluorescent, phosphorescent, photochromic, and/or thermochromic and is visible under excitation by UV, infrared radiation, or by specific optics; or the at least one marker has electrical, magnetic, or electromagnetic properties and is detectable under excitation by radiofrequency vibration or at an ultrasonic frequency.

44. A method for revealing attempts at forgery of a security substrate as claimed in claim 22, wherein
the security substrate or a security document containing the security substrate comprises at least one area for authentication and/or for revealing attempts at forgery using solvents, the at least one area comprising first individualized polymer- or copolymer-based particles comprising at least one first marker, the polymer or copolymer being at least partially soluble in solvents used for forgery,
wherein the at least one first marker is visible or detectable, and
wherein the at least one first marker is iridescent, luminescent, fluorescent, phosphorescent, photochromic, and/or thermochromic and is visible under excitation by UV, infrared radiation, or by specific optics; or the at least one marker has electrical, magnetic, or electromagnetic properties and is detectable under excitation by radiofrequency vibration or at an ultrasonic frequency,
the method comprising:
verifying whether the at least one first marker is invisible or undetectable so as to reveal an attempt at forgery of the security substrate or of the security document using solvents, or so as to conclude that the security substrate or the security document is not authentic,
or
verifying whether the at least one first marker is visible but has a different appearance or is detectable but exhibits a different signal so as to reveal an attempt at forgery of the security substrate or of the security document using solvents.

45. The method of claim 44, wherein the security substrate or the security document containing the security substrate comprises second individualized polymer- or copolymer-based particles comprising at least one second marker in the at least one area, the polymer or copolymer of the second individualized particles being insoluble in solvents used for forgery and the at least one second marker being visible or detectable, and the method further comprising:
verifying whether the at least one second marker is visible or detectable so as to reveal an attempt at forgery of the security substrate or of the security document using solvents.

46. A method for manufacturing a security substrate as claimed in claim 22, the method comprising:
introducing the individualized polymer- or copolymer-based particles comprising the at least one marker into a body of the security substrate; and/or
applying the individualized polymer- or copolymer-based particles comprising the at least one marker at a surface of the security substrate by at least one of layering, coating, impregnating, and printing the individualized particles on the surface of the security substrate.

47. The method of claim 46, further comprising randomly dispersing the individualized polymer- or copolymer-based particles comprising the at least one marker in a fibrous suspension before or during formation of the security substrate in sheets so as to randomly introduce the individualized particles into the body of the security substrate.

48. The method of claim 46, further comprising randomly mixing the individualized polymer- or copolymer-based particles comprising the at least one marker with a structure made of synthetic material or randomly applying the individualized polymer- or copolymer-based particles comprising the at least one marker to a structure made of synthetic material, wherein the structure has voids, and wherein the random mixing or applying occurs before coextruding and/or bi-stretching the synthetic structure, or
randomly applying, at the surface of the substrate, the individualized polymer- or copolymer-based particles comprising the at least one marker to the synthetic structure after coextruding and/or bi-stretching the synthetic structure.

49. The method of claim 48, further comprising placing an adhesive layer on the security substrate, and wherein the synthetic structure is made of polyethylene having voids with a density of less than about 0.6 g/cm³.

50. The method of claim 49, wherein the synthetic structure has a cohesive strength of less than about 4 Newtons.
